(19) United States
(12) Patent Application Publication
(10) Pub. No.: US 2009/0198157 A1
(43) Pub. Date: Aug. 6, 2009

(54) ULTRASOUND MOXIBUSTION METHOD AND DEVICE

(76) Inventor: Eilaz Babaev, Minnetonka, MN (US)

Correspondence Address: Bacoustics, LLC, 5929 BAKER ROAD, SUITE 470, MINNETONKA, MN, 55345, US

(21) Appl. No.: 12/024,521

(22) Filed: Feb. 1, 2008

(51) Int. Cl. A61N 7/02

(52) U.S. Cl. 601/3

(57) ABSTRACT

The present invention is directed towards a device and methods for the use of ultrasonic energy for providing therapeutic effects by heating and stimulating bioactive points (BAPs) in the body of a patient. The device includes an ultrasound generator driving an ultrasound transducer. The ultrasound transducer generates ultrasound waves which are focused to a focal point through a lens which is attached to the ultrasound generator. A coupling medium is used displace any air between the lens and the patient's skin surface to allow ultrasonic energy transfer into the patient. The device is pressed against the patient's skin surface and the unit is energized using a foot or hand activated switch.

### BACKGROUND OF THE INVENTION

The invention relates to a method and device for activating acupuncture or bioactive points (BAPs) points of a body and, more particularly, for stimulating BAPs within a body using ultrasound energy applied through the skin surface without physically breaking the skin surface with needles or other devices. The invention provides heat and stimulation to achieve similar therapeutic results and benefits that have been obtained by the practice of acupuncture, acupressure and more specifically moxibustion as practiced in eastern countries for centuries without the risk of infection or burns which are associated with the historical practices.

Traditionally, acupuncture, acupressure and moxibustion relate to the stimulation of bioactive points (BAPs) along the body meridians. Energy flow along the body meridians is believed to be important for good health, and if a BAP is not active, energy flow stops at this point. This is believed to have a negative health impact which can produce a number of symptoms. Hundreds of BAPs have been described and documented in the prior art. Activation of a typical BAP may require from minutes to hours with times of 10 to 20 minutes typically being required for each location. Because of this time requirement, it is impractical to treat large numbers of BAPs on a patient during a typical treatment session. Generally, 10 to 15 key BAPs have been identified within a body that are believed to be of special significance. Adequate stimulation to a specific BAP may result in particular health or medical effects, such as relieving pain and enhancing metabolism, to improve human health.

Stimulation of specific acupuncture points has been shown in several studies to have utility as adjuvant therapy to conventional medical treatments such as nausea and vomiting, postoperative pain, headache, smoking cessation, erectile dysfunction, depression, male fertility, dysmenorrhea, and stomach acid secretion. Furthermore acupuncture may be useful as an adjunct treatment or an acceptable alternative or be included in a comprehensive management program in adult postoperative and chemotherapy nausea and vomiting and in postoperative dental pain. Other health issues that have been reported to benefit from acupuncture/moxibustion therapy include; addiction, stroke rehabilitation, headache, menstrual cramps, tennis elbow, fibromyalgia, myofascial pain, osteoarthritis, low back pain, carpal tunnel syndrome, and asthma.

Acupuncture is an ancient medical art dating back more than 2000 years. Traditionally, BAP's in the human body were reached by piercing the body with fine wires or needles. Acupuncture includes the stimulation of the BAPs with needles which are placed through the skin to a proper depth at the bioactive points and may be twisted or rotated about their longitudinal axes by the thumb and finger of the acupuncturist to assist the activation.

Acupuncture and moxibustion are an important component of traditional Chinese medicine with a long history. Acupressure relies on external pressure administered to activate the BAPs, while moxibustion relies on the production of heat to stimulate the BAP and restore the blocked energy flow.

Moxibustion may be administered by providing direct heat or infrared radiation to a BAP. Specific examples include holding the lighted end of a cigar or cigarette shaped roll of dried leaves and herbs next to the BAP but away from the skin to prevent burning. Even so, burning of skin is a common occurrence from moxibustion. Moxibustion may also be used in combination with acupuncture where the needle inserted to the BAP is heated with a heat source, thereby warming the needle which heats the BAP. Sterile techniques, high skill levels and considerable training are required for the moxibustion/acupuncture practitioner to avoid negative complications of the procedures. Even with the use of needles, some BAP's are located so deep beneath the skin that they are difficult or impractical to access through conventional methods.

In addition, many patients resist acupuncture due to a fear of its needling or a contraindication for acupuncture using acupuncture needles (e.g., bleeding disorders, active infection). The needle phobic pediatric patient, in particular, is extremely reluctant. Therefore, an effective means of stimulation of BAPs from the skin or surface of a body without invasive needles is needed.

### BRIEF SUMMARY OF THE INVENTION

The present invention is directed towards a device and method for the use of ultrasonic energy for providing therapeutic effects by heating and stimulating bioactive points (BAPs) in the body of a patient. The device includes an ultrasound generator driving an ultrasound transducer. The ultrasound transducer is located in a housing designed to assist operator control of the device. The ultrasound transducer generates ultrasound waves which pass through the lens which is attached to the ultrasound transducer. The ultrasound waves are focused by and are focused to a focal point. A coupling medium is used to displace any air between the lens and the patient's skin surface to allow ultrasonic energy transfer from the ultrasound transducer into the patient.

A lens with a focal point appropriate for the depth of the BAP is selected and attached to the ultrasound transducer. The device is pressed against the patient's skin surface and the unit is energized using a foot or hand activated switch. After a period of time, preferably from 5-10 seconds, the unit is deactivated and moved to another location. Focusing the ultrasonic energy provides a very rapid means to heat and stimulate the specific areas of the BAPs without impacting surrounding areas.

An object of the invention is to provide hyperthermic and vibratory simulation of BAPs without the use of needles or other invasive techniques.

Another object of the invention is to supply controlled heat to BAPs without the use of heated devices in close proximity to the skin.

Another object of the invention is to provide a moxibustion method that does not require extensive practitioner training.

Another object of the invention is to provide a method of BAP stimulation that can be done faster than existing procedures.

Another object of the invention is to provide a moxibustion device that can simulate deep BAP's that may be located up to 2 to 3 inches below the surface of the skin.

Another object of the invention is to provide a moxibustion device that can stimulate a BAP without affecting surrounding tissue.

### BRIEF DESCRIPTION OF THE DRAWINGS

FIG. 1 is an illustration of a hand held embodiment of the moxibustion

FIG. 2 is an illustration of another embodiment of the moxibustion device lacement.

FIG. 3 is an illustration of an embodiment of the moxibustion device into an array.

### DETAILED DESCRIPTION OF THE INVENTION

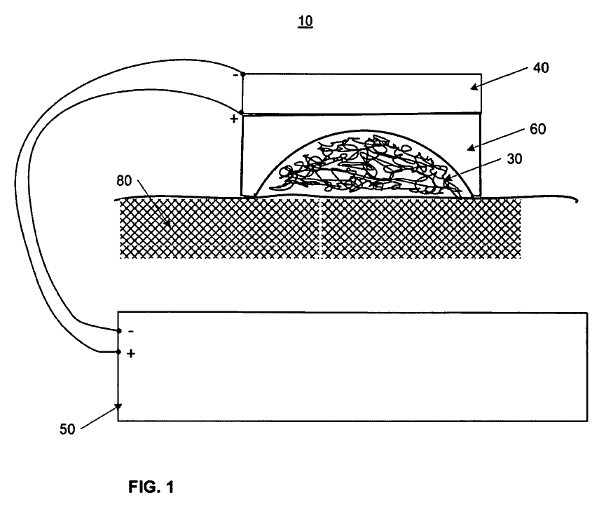

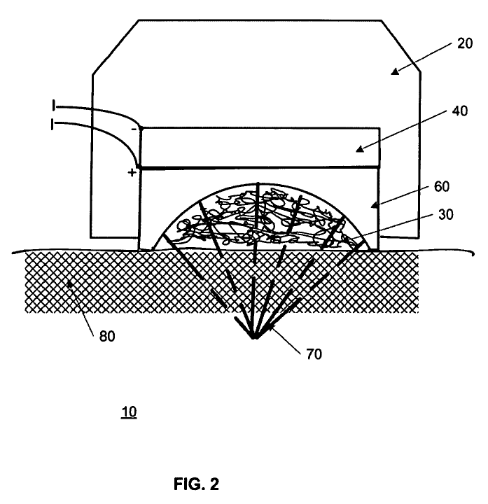

The present invention is directed towards a device 10 and methods for the use of ultrasonic energy for providing therapeutic effects by heating and stimulating bioactive points (BAPs) beneath the skin 80 of a patient. As shown in FIG. 1 and FIG. 2, the device 10 includes an ultrasound generator 50 typically powered with standard AC current which is electrically connected to an ultrasound transducer 40. The ultrasound generator 50 may produce a signal form that can be sinusoidal, trapezoidal or rectangular. The rectangular signal form embodiment is preferred with the present invention.

The ultrasound transducer 40 is located in a housing 20 designed to assist operator control of the device 10. The housing 20 may be a hand piece style design for easy grasping and manipulation. As another embodiment, the ultrasound transducer 40 may be affixed to a web material to provide close contact to the patient's body with straps, Velcro bands or similar mechanisms.

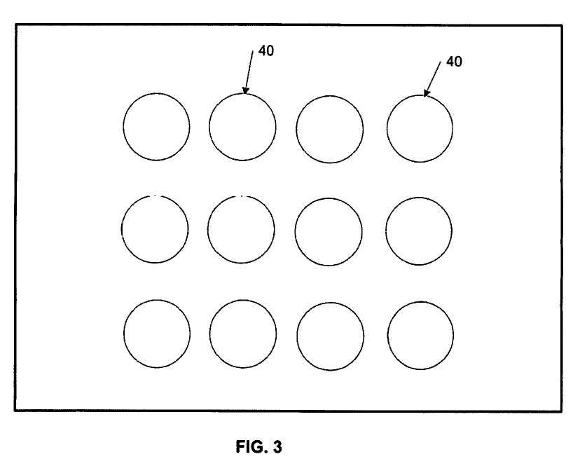

The ultrasound transducer 40 may be of the Langevin or cymbal style transducer. The cymbal style transducer is preferred because of its compact profile due to its wafer shape and correspondingly relatively low cost. Several transducers may be arranged into the form of an array as shown in FIG. 3. This allows the ability of focusing multiple transducers on a specific BAP or in rapid sequence without relocating the device 10. Alternatively, this embodiment may be used for simultaneously activating multiple BAPs.

The ultrasound transducers 40 may be operated to produce an ultrasound wave preferably in the range of 1 to 5 mHz, with 1 mHz generally providing the optimal therapeutic combination of heat and vibration. The wavelength of the ultrasound waves 70 is preferrably no larger than 2 to 3 millimeters to allow precise focusing on specific BAPs.

The ultrasound transducer 40 generates ultrasound waves 70 which are focused to a focal point through a lens 60 which is attached to the ultrasound transducer 40. For effective immediate and localized stimulation and immediate heating, vibration and activation of the specific BAPs the focal point of the lens 60 is imposed on the BAP. The lens 60 is preferable concave with a focal length typically between 5 and 20 millimeter, but the lens 60 could have a focal length between 2 to 3 millimeters for activating sites just beneath the skin 80 to up to 80 millimeters for activating sites in deeper tissues such as deep within muscle tissue of a patient's thighs. It is preferable if the focal point of the lens 60 is known to within 1-millimeter for proper selection for a particular use.

Generally, there is an air gap between the concave surface of the lens 60 and the patient's skin surface. Since ultrasound waves 70 would be reflected off any air interface between the patient and the transducer, it is important to utilize a coupling medium 30 to displace even small air spaces between the lens 60 and the patient's skin surface. A liquid or gel may be used to avoid the reflection of the ultrasound waves 70 off the solid/air interface and to allow ultrasonic energy transfer from the ultrasound transducer 40 into the patient. Due to its convenience and ease of use, a gel pack designed to fit within the air space between the lens 60 and the patient's skin is preferred. The gel pack being a disposable sealed bag that contains the liquid or gel that is utilized to fill the air gap within the concave surface of the lens 60.

A lens 60 with a focal point appropriate for the depth of the BAP is selected and attached to the ultrasound transducer 40. The device 10 is pressed against the patient's skin surface and is either mechanically secured or simply held against the patient's skin 80. The unit is energized preferably using a foot or hand activated switch. After a period of time, preferably from 5-10 seconds, the ultrasound transducer 40 is either manually or automatically deactivated and the moved to the next BAP location.

Focusing the ultrasonic energy provides a very rapid means to heat and stimulate the specific areas of the BAPs without impacting surrounding areas. To control and limit temperature rise within the tissue, the ultrasound generator 50 may be programmable to provide a rapid pulsed on-off signal to the ultrasound transducer 40. This pulsed signal may vary between 0 to 100% on, with 20% on being the preferred percentage on time.

Under the present invention, only ultrasonic energy is being transferred across the patient's skin boundary, therefore, the invention can provide hyperthermic and vibratory stimulation of BAPs without the use of needles or other invasive techniques. Since this invention focuses the ultrasound energy at the focal point, the invention does not require direct heat in close proximity to the skin 80.

The present invention can stimulate BAP's that may be located up to 2 to 3 inches below the surface of the skin which are inaccessible using current procedures. The device 10 can stimulate a BAP without affecting surrounding tissue. Furthermore, moxibustion using the invention can be completed in much less time than required under existing protocols. Also, since the invention does not use invasive techniques, the invention can be administered by practitioners that do not need to be trained in sterile techniques.

It should be appreciated that elements described with singular articles such as “a”, “an”, and/or “the” and/or otherwise described singularly may be used in plurality. It should also be appreciated that elements described in plurality may be used singularly.

Although specific embodiments of apparatuses and methods have been illustrated and described herein, it will be appreciated by those of ordinary skill in the art that any arrangement, combination, and/or sequence of that is calculated to achieve the same purpose may be substituted for the specific embodiments shown. It is to be understood that the above description is intended to be illustrative and not restrictive. Combinations of the above embodiments and other embodiments as well as combinations and sequences of the above methods and other methods of use will be apparent to individuals possessing skill in the art upon review of the present disclosure.

The scope of the claimed apparatus and methods should be determined with reference to the appended claims, along with the full scope of equivalents to which such claims are entitled.

What is claimed is:

1. A device for administering moxibustion using noninvasive ultrasonic energy comprising:
a housing securing an ultrasound transducer for generating ultrasound waves; and
a lens for focusing the ultrasound waves through a coupling medium.

2. The device of claim 1 wherein the ultrasound transducer is a cymbal transducer.

3. The device of claim 1 wherein the coupling medium is contained within a gel pack.

4. The device of claim 1 wherein the ultrasound transducer is driven by a rectangular signal form.

5. The device of claim 1 wherein a plurality of ultrasound transducers are arranged into an array.

6. The device of claim 5 wherein the ultrasound transducer is activated through a programmable controller.

7. The device of claim 6 wherein the ultrasound transducers are activated sequentially.

8. The device of claim 6 wherein the ultrasound transducers are activated simultaneously.

9. A method for administering moxibustion using noninvasive ultrasonic energy comprising the steps of:
obtaining a device having a housing securing an ultrasound transducer for generating ultrasound waves and a lens fixed to the ultrasound transducer;
placing a coupling medium between the lens and a patient's skin surface;
securing the device to the patient's skin surface;
activating the ultrasound transducer to produce the ultrasound waves;
focusing the ultrasound waves through the coupling medium into the patient; and
heating and vibrating a bioactive point to achieve a therapeutic effect.

10. The method of claim 9 wherein the step of heating and vibrating a bioactive point to achieve a therapeutic effect is between 5 and 10 seconds long.

11. The method of claim 9 wherein the ultrasound waves have a frequency of 1 mHz.

12. The method of claim 9 wherein the bioactive point is located within the range between 2 and 80 millimeters beneath the patient's skin surface.

13. The method of claim 9 wherein the coupling medium is a gel pack.

14. The method of claim 9 wherein the ultrasound transducer is a cymbal transducer.

15. The method of claim 9 having a plurality of ultrasound transducers are arranged into an array.

16. The device of claim 15 wherein the array is activated through a programmable controller.

17. The device of claim 16 wherein the ultrasound transducers are activated sequentially to treat a plurality of bioactive points.

18. The device of claim 16 wherein the ultrasound transducers are activated simultaneously to treat a plurality of bioactive points.
